(19) United States
(12) Patent Application Publication
(10) Pub. No.: US 2023/0380756 A1
(43) Pub. Date: Nov. 30, 2023

(54) BED WITH USER TRACKING FEATURES

(71) Applicant: Sleep Number Corporation, Minneapolis, MN (US)

(72) Inventors: Wade Daniel Palashewski, Andover, MN (US); Gordan Redzic, Park Ridge, IL (US); Kelley Parker, Robbinsdale, MN (US)

(21) Appl. No.: 18/204,141

(22) Filed: May 31, 2023

Related U.S. Application Data
(63) Continuation of application No. 16/998,420, filed on Aug. 20, 2020.
Application No. 16/998,420 is a continuation of application No. 14/988,448, filed on Jan. 5, 2016, now abandoned.
(60) Provisional application No. 62/099,907, filed on Jan. 5, 2015.

(51) Int. Cl. A61B 5/00; G06N 5/025; A47C 27/08; A47C 27/10; A47C 31/00

(52) U.S. Cl. CPC A61B 5/4806; G06N 5/025; A47C 27/082; A47C 27/083; A47C 27/10; A47C 31/008; A61B 5/4809; A61B 5/4815; A61B 5/1116

(57) ABSTRACT

A computer application interface is provided, and receives first input indicating that the user has completed a first task. It is determined that the user is assigned to a second task. A bed output event and a bed output device are selected. The bed output event includes instructions for a bed output device. The bed output device is physically coupled to a bed and capable of responding to instructions to generate some output. The selected bed output event and bed output device are selected to facilitate the second task.

### CROSS-REFERENCE TO RELATED APPLICATIONS

This application is a continuation application of U.S. application Ser. No. 16/998,420, filed on Aug. 20, 2020, which is a continuation application U.S. application Ser. No. 14/988,488, filed on Jan. 5, 2016, which claims priority to pending U.S. Provisional Application Ser. No. 62/099,907, entitled “Bed with User Tracking Features,” filed on Jan. 5, 2015, the entire contents of which are incorporated herein by reference.

The present document relates to a bed with user tracking features.

### BACKGROUND

A computer network is a collection of computers and other hardware interconnected by communication channels that allow sharing of resources and information. Communication protocols define the rules and data formats for exchanging information in a computer network.

In general, a bed is a piece of furniture used as a location to sleep or relax. Many modern beds include a soft mattress on a bed frame. The mattress may include springs, foam material, and/or an air bladder to support the weight of one or more occupants.

### SUMMARY

In one aspect, a method performed by data processing apparatuses, the method includes providing, to a user, a computer application interface. The method further includes receiving, from the user through the computer application interface, first input indicating that the user has completed a first task. The method further includes determining, based on the first input, that the user is assigned to a second task. The method further includes selecting, based on the second task, a bed output event and a bed output device. The bed output event includes instructions for a bed output device. The bed output device is physically coupled to a bed and capable of responding to instructions to generate some output. The selected bed output event and bed output device are selected to facilitate the second task. The method further includes causing the selected output device to perform the selected bed output event.

Implementations can include any, all, or none of the following features. The method including receiving, from the user, second input to a bed input device that is capable of detecting at least one physical phenomena associated with the bed; and wherein determining that the user is assigned to a second task is further based on the second input. The bed input device is communicably coupled to a bed and physically coupled to the bed by a data plug. Determining that the user is assigned to a second task is further based on a detection of a reduction in light at the light sensor. The method includes a plurality of tasks associated with a bedtime routine of the user; the first input is an indication of completion of a first task of the plurality of tasks of the checklist; and the second task is another of the plurality of tasks of the checklist. The plurality of tasks of the checklist is an ordered list; and the first task precedes the second task in the ordered list and at least one addition tasks is in the ordered list between the first task and the second task. The computer application interface includes a video game; the first input is an indication of completion of a goal within the video game; and the second task is a game element unavailable to the user before completion of the goal within the video game. The bed output device includes under-bed lighting; and the bed output event includes a command to illuminate the under-bed lighting. The second task is turning off a light switch. The bed output device includes an inflatable bed-elevation bladder; and the bed output event includes a command to inflate the inflatable bed-elevation bladder. The second task is reading a book. At least some of the bed output devices are communicably coupled to a bed and physically coupled to the bed by a data plug. The bed includes a mattress that includes an air bladder with a pressure sensor fluidically connected to the air bladder. The method including providing, to the user, a second computer application interface; receiving, from the user through the second computer application interface, third input indicating that the user has completed the first task; determining, based on the first input, that the user is assigned to the second task; selecting, based on the second task, the bed output event and the bed output device; and causing the selected output device to perform the selected bed output event. The computer application interface is designed for display to users of a first age; the second computer application interface is designed for display to users of a second age older than the first age; and providing, to the user, a second computer application interface includes determining that the user has passed an age threshold.

In one aspect, a system includes an interface device configured to: provide, to a user, a computer application interface. The interface device is further configured to receive, from the user, first input indicating that the user has completed a first task. The system further includes a data processing device configured to: determine, based on the first input, that the user is assigned to a second task. The data processing device is further configured to select, based on the second task a bed output event and a bed output device. The bed output event includes instructions for a bed output device. The bed output device is physically coupled to a bed and capable of responding to instructions to generate some output. The selected bed output event and bed output device are selected to facilitate the second task. The system further includes a bed output device configured to perform the selected bed output event.

In one aspect, a device configured to provide, to a user, a computer application interface. The device is further configured to receive, from the user, first input indicating that the user has completed a first task. The device is further configured to determine, based on the first input, that the user is assigned to a second task. The device is further configured to select, based on the second task a bed output event and a bed output device. The bed output event includes instructions for a bed output device. The bed output device is physically coupled to a bed and capable of responding to instructions to generate some output. The selected bed output event and bed output device are selected to facilitate the second task. The device is further configured to cause the selected output device to perform the selected bed output event.

In one aspect, a method is performed by data processing apparatuses, the method includes maintaining, in computer memory, a profile for a user, the profile includes data for a plurality of features of a computer application. The method further includes receiving, from the user, input to a bed input device indicating at least presence of a person on the bed. The method further includes determining, from the input, a sleep parameter that represents a measure of the presence of the person on the bed. The method further includes comparing the sleep parameter to a rule-set to determine if the sleep parameter meets a test condition of the rule-set. The method further includes responsive to determining that the sleep parameter meets a test condition of the rule-set, modifying the data for at least one of the features of the computer application.

Implementations can include any, all, or none of the following features. The computer application is a video game; the sleep parameter is a duration of bed occupancy comparing the sleep parameter to a rule-set includes determining if the sleep parameter indicates a duration of bed occupancy greater than a threshold value; and modifying the data for at least one of the features of the computer application includes crediting the profile with a game resource. The game resource is at least one of the group consisting of in-game currency, time-based access to the game, an interactive features, a badge, and an in-game statistic. The input to the bed input device indicates at least presence of a person on the bed for a duration beginning at a first time and ending at a second time that is after the first time; and the data for at least one of the features of the computer application is modified at a third time that is after the second time. The method including responsive to determining that the sleep parameter meets a test condition of the rule-set, outputting a report indicating that the sleep parameter meets a test condition of the rule-set. The method including responsive to determining that the sleep parameter does not meet a test condition of the rule-set, executing coaching-based content. The coaching-based content is directed to encouraging corrected behavior regarding the test condition.

In one aspect, a method is performed by data processing apparatuses, the method includes maintaining, in computer memory, a profile for a user, the profile includes data for a plurality of features of a computer application. The method further includes receiving, from the user, input to a bed input device indicating at least presence of a person on the bed. The method further includes determining, from the input, a sleep parameter that represents a measure of the presence of the person on the bed. The method further includes comparing the sleep parameter to a rule-set to determine if the sleep parameter fails a test condition of the rule-set. The method further includes responsive to determining that the sleep parameter meets a test condition of the rule-set, modifying the data for at least one of the features of the computer application to remove user access to the application.

The systems and processes described here may be used to provide a number of potential advantages. By tracking the state of a user in their bedtime routine, a computer system can cause a bed with an output device to engage the output device in preparation for the user. If the bed also has an input device, the input device may be included in the user state tracking. As such, a bed and related output devices may make for a more pleasant user experience when a person is performing their bedtime routine. When a user sleeps, their sleep activity may be monitored. This sleep activity may be used as the basis of a reward system that credits a user when they sleep well. For example, a child may be rewarded with unlocked video game time the next day if they stay in their bed the night before.

Other features, aspects and potential advantages will be apparent from the accompanying description and figures.

### DESCRIPTION OF DRAWINGS

FIG. 1 shows an example air bed system.

FIG. 2 is a block diagram of various components of the air bed system of FIG. 1, according to an example.

FIG. 3 shows an example environment including a bed in communication with devices located in and around a home.

FIG. 4 is an example computer interface showing a bedtime checklist application.

FIG. 5 is an example computer interface showing a bedtime videogame application.

FIG. 6 is a swimlane diagram of an example process for tracking a user's state and updating output devices.

FIG. 7 is a swimlane diagram of an example process for tracking a user's state and updating a profile associated with the user.

FIG. 8 is a schematic diagram that shows an example of a computing device and a mobile computing device.

FIGS. 9 and 10 are example computer interfaces showing awards that are awarded to a user profile in response to input received from bed-based sensors.

Like reference symbols in the various drawings indicate like elements

### DETAILED DESCRIPTION

A bed system can include input devices (e.g., light sensors, pressure sensors), output devices (e.g., air pumps, under-bed illumination) and a control system that is able to monitor the input devices and control the output devices. The control system may track one or more bed users and have personalized accounts for each such user. The status of each user may be monitored and predicted by the control system, and the output devices of the bed may be manipulated in accordance with the predicted state of the user.

In one instance, this technology may be utilized to assist in the bed-time routine of a child. For example, the control system may have an interface screen that shows the child a bedtime checklist or game. This interface can guide the child through their bedtime routine, preparing the bed outputs as needed and tracking the child through their inputs to the interface and the other bed inputs. For example, upon completion of reading a book, the control system may predict that the child will be turning off the light switch in their room. To facilitate this activity, the control system may turn on an under-bed illumination output device for five minutes.

In another instance, the child can be rewarded in their personal profile for some targeted behavior. For example, if the child sleeps in their bed all night, as determined by an air-pressure sensor input device, their account may be credited with twenty minutes of access to video games associated with their account, or their account may be credited with in-game virtual currency.

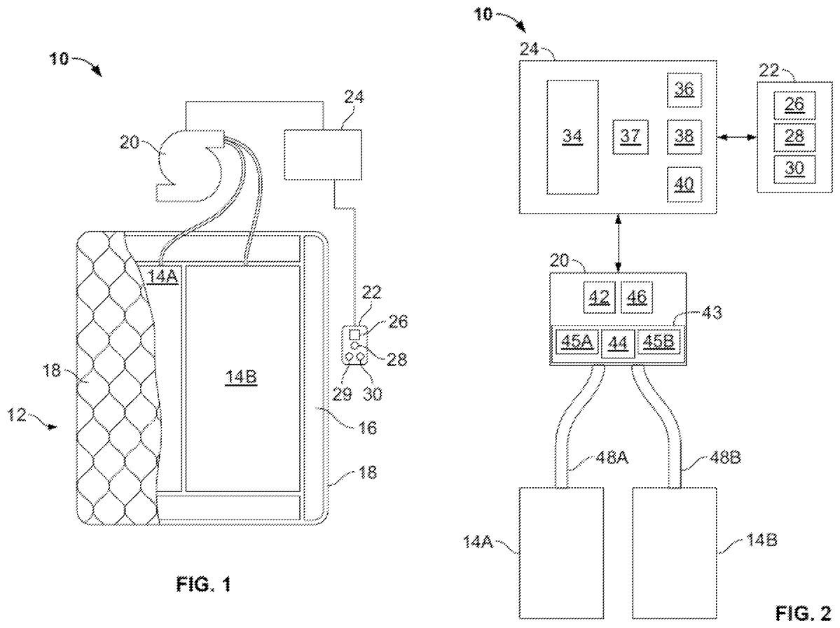

FIG. 1 shows an example air bed system 10 that includes a bed 12. The bed 12 includes at least one air chamber 14 surrounded by a resilient border 16 and encapsulated by bed ticking 18. The resilient border 16 may comprise any suitable material, such as foam.

As illustrated in FIG. 1, the bed 12 can be a two chamber design having first and second fluid chambers, such as a first air chamber 14A and a second air chamber 14B. In alternative embodiments, the bed 12 can include chambers for use with fluids other than air that are suitable for the application. In some embodiments, such as single beds or kids' beds, the bed 12 can include a single air chamber 14A or 14B or multiple air chambers 14A and 14B. First and second air chambers 14A and 14B can be in fluid communication with a pump 20. The pump 20 can be in electrical communication with a remote control 22 via control box 24. The control box 24 can include a wired or wireless communications interface for communicating with one or more devices, including the remote control 22. The control box 24 can be configured to operate the pump 20 to cause increases and decreases in the fluid pressure of the first and second air chambers 14A and 14B based upon commands input by a user using the remote control 22. In some implementations, the control box 24 is integrated into a housing of the pump 20.

The remote control 22 may include a display 26, an output selecting mechanism 28, a pressure increase button 29, and a pressure decrease button 30. The output selecting mechanism 28 may allow the user to switch air flow generated by the pump 20 between the first and second air chambers 14A and 14B, thus enabling control of multiple air chambers with a single remote control 22 and a single pump 20. For example, the output selecting mechanism 28 may by a physical control (e.g., switch or button) or an input control displayed on display 26. In some cases, the remote control 22 may be incorporated into another device such as a smart phone or tablet computer. Separate remote control units can be provided for each air chamber and may each include the ability to control multiple air chambers. Pressure increase and decrease buttons 29 and 30 may allow a user to increase or decrease the pressure, respectively, in the air chamber selected with the output selecting mechanism 28. Adjusting the pressure within the selected air chamber may cause a corresponding adjustment to the firmness of the respective air chamber. In some embodiments, the remote control 22 can be omitted or modified as appropriate for an application. For example, in some embodiments the bed 12 can be controlled by a computer, tablet, smart phone, or other device in wired or wireless communication with the bed 12.

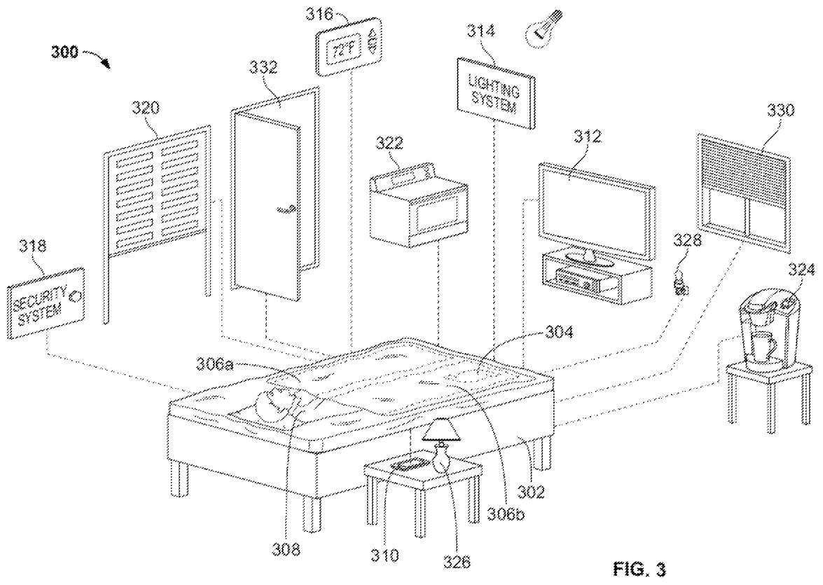

FIG. 2 is a block diagram detailing data communication between certain components of the example air bed system 10 according to various examples. As shown in FIG. 2, the control box 24 may include a power supply 34, a processor 36, a memory 37, a switching mechanism 38, and an analog to digital (A/D) converter 40. The switching mechanism 38 can be, for example, a relay or a solid state switch. In some implementations, the switching mechanism 38 can be located in the pump 20 rather than the control box 24.

The pump 20 and the remote control 22 are in two-way communication with the control box 24. The pump 20 includes a motor 42, a pump manifold 43, a relief valve 44, a first control valve 45A, a second control valve 45B, and a pressure transducer 46. The pump 20 is fluidly connected with the first air chamber 14A and the second air chamber 14B via a first tube 48A and a second tube 48B, respectively. The first and second control valves 45A and 45B can be controlled by switching mechanism 38, and are operable to regulate the flow of fluid between the pump 20 and first and second air chambers 14A and 14B, respectively.

In some implementations, the pump 20 and the control box 24 can be provided and packaged as a single unit. In some alternative implementations, the pump and the control box 24 may be provided as physically separate units. In some implementations, the control box 24, the pump 20, or both are integrated within or otherwise contained within a bed frame or bed support structure that supports the bed 12. In some implementations, the control box 24, the pump 20, or both are located outside of a bed frame or bed support structure (as shown in the example in FIG. 1).

The example air bed system 10 depicted in FIG. 2 includes the two air chambers 14A and 14B and the single pump 20. However, other implementations may include an air bed system having two or more air chambers and one or more pumps incorporated into the air bed system to control the air chambers. For example, a separate pump can be associated with each air chamber of the air bed system or a pump can be associated with multiple chambers of the air bed system. Separate pumps may allow each air chamber to be inflated or deflated independently and simultaneously. Furthermore, additional pressure transducers may also be incorporated into the air bed system such that, for example, a separate pressure transducer can be associated with each air chamber.

In use, the processor 36 can, for example, send a decrease pressure command to one of air chambers 14A or 14B, and the switching mechanism 38 can be used to convert the low voltage command signals sent by the processor 36 to higher operating voltages sufficient to operate the relief valve 44 of the pump 20 and open the control valve 45A or 45B. Opening the relief valve 44 may allow air to escape from the air chamber 14A or 14B through the respective air tube 48A or 48B. During deflation, the pressure transducer 46 may send pressure readings to the processor 36 via the A/D converter 40. The A/D converter 40 may receive analog information from pressure transducer 46 and may convert the analog information to digital information useable by the processor 36. The processor 36 may send the digital signal to the remote control 22 to update the display 26 in order to convey the pressure information to the user.

As another example, the processor 36 can send an increase pressure command. The pump motor 42 can be energized in response to the increase pressure command and send air to the designated one of the air chambers 14A and 14B through the air tube 48A or 48B via electronically operating the corresponding valve 45A or 45B. While air is being delivered to the designated air chamber 14 A or 14B in order to increase the firmness of the chamber, the pressure transducer 46 may sense pressure within the pump manifold 43. Again, the pressure transducer 46 may send pressure readings to the processor 36 via the A/D converter 40. The processor 36 may use the information received from the A/D converter 40 to determine the difference between the actual pressure in air chamber 14A or 14B and the desired pressure. The processor 36 may send the digital signal to the remote control 22 to update display 26 in order to convey the pressure information to the user.

Generally speaking, during an inflation or deflation process, the pressure sensed within the pump manifold 43 can provide an approximation of the pressure within the respective air chamber that is in fluid communication with the pump manifold 43. An example method of obtaining a pump manifold pressure reading that is substantially equivalent to the actual pressure within an air chamber includes turning off pump 20, allowing the pressure within the air chamber 14A or 14B and the pump manifold 43 to equalize, and then sensing the pressure within the pump manifold 43 with the pressure transducer 46. Thus, providing a sufficient amount of time to allow the pressures within the pump manifold 43 and chamber 14A or 14B to equalize may result in pressure readings that are accurate approximations of the actual pressure within air chamber 14A or 14B. In some implementations, the pressure of the air chambers 14A and/or 14B can be continuously monitored using multiple pressure sensors.

In some implementations, information collected by the pressure transducer 46 can be analyzed to determine various states of a person lying on the bed 12. For example, the processor 36 can use information collected by the pressure transducer 46 to determine a heart rate or a respiration rate for a person lying in the bed 12. For example, a user can be lying on a side of the bed 12 that includes the chamber 14A. The pressure transducer 46 can monitor fluctuations in pressure of the chamber 14A and this information can be used to determine the user's heart rate and/or respiration rate. As another example, additional processing can be performed using the collected data to determine a sleep state of the person (e.g., awake, light sleep, deep sleep). For example, the processor 36 may determine when a person falls asleep and, while asleep, the various sleep states of the person.

Additional information associated with a user of the bed system 10 that can be determined using information collected by the pressure transducer 46 includes motion of the user, presence of the user on a surface of the bed 12, weight of the user, heart arrhythmia of the user, and apnea. Taking user presence detection for example, the pressure transducer 46 can be used to detect the user's presence on the bed 12, e.g., via a gross pressure change determination and/or via one or more of a respiration rate signal, heart rate signal, and/or other biometric signals. For example, a simple pressure detection process can identify an increase in pressure as an indication that the user is present in the bed 12. As another example, the processor 36 can determine that the user is present in the bed 12 if the detected pressure increases above a specified threshold (so as to indicate that a person or other object above a certain weight is positioned on the bed 12). As yet another example, the processor 36 can identify an increase in pressure in combination with detected slight, rhythmic fluctuations in pressure as corresponding to the user being present on the bed 12. The presence of rhythmic fluctuations can be identified as being caused by respiration or heart rhythm (or both) of the user. The detection of respiration or a heartbeat can distinguish between the user being present on the bed and another object (e.g., a suit case) being placed upon the bed.

In some implementations, fluctuations in pressure can be measured at the pump 20. For example, one or more pressure sensors can be located within one or more internal cavities of the pump 20 to detect fluctuations in pressure within the pump 20. The fluctuations in pressure detected at the pump 20 can indicate fluctuations in pressure in one or both of the chambers 14A and 14B. One or more sensors located at the pump can be in fluid communication with the one or both of the chambers 14A and 14B, and the sensors can be operative to determine pressure within the chambers 14A and 14B. The control box 24 can be configured to determine at least one vital sign (e.g., heart rate, respiratory rate) based on the pressure within the chamber 14A or the chamber 14B.

In some implementations, the control box 24 can analyze a pressure signal detected by one or more pressure sensors to determine a heart rate, respiration rate, and/or other vital signs of a user lying or sitting on the chamber 14A or the chamber 14B. More specifically, when a user lies on the bed 12 positioned over the chamber 14A, each of the user's heart beats, breaths, and other movements can create a force on the bed 12 that is transmitted to the chamber 14A. As a result of the force input to the chamber 14A from the user's movement, a wave can propagate through the chamber 14A and into the pump 20. A pressure sensor located at the pump 20 can detect the wave, and thus the pressure signal output by the sensor can indicate a heart rate, respiratory rate, or other information regarding the user.

With regard to sleep state, system 10 can determine a user's sleep state by using various biometric signals such as heart rate, respiration, and/or movement of the user. While the user is sleeping, the processor 36 can receive one or more of the user's biometric signals (e.g., heart rate, respiration, and motion) and determine the user's present sleep state based on the received biometric signals. In some implementations, signals indicating fluctuations in pressure in one or both of the chambers 14A and 14B can be amplified and/or filtered to allow for more precise detection of heart rate and respiratory rate.

The control box 24 can perform a pattern recognition algorithm or other calculation based on the amplified and filtered pressure signal to determine the user's heart rate and respiratory rate. For example, the algorithm or calculation can be based on assumptions that a heart rate portion of the signal has a frequency in the range of 0.5-4.0 Hz and that a respiration rate portion of the signal a has a frequency in the range of less than 1 Hz. The control box 24 can also be configured to determine other characteristics of a user based on the received pressure signal, such as blood pressure, tossing and turning movements, rolling movements, limb movements, weight, the presence or lack or presence of a user, and/or the identity of the user. Techniques for monitoring a user's sleep using heart rate information, respiration rate information, and other user information are disclosed in U.S. Patent Application Publication No. 20100170043 to Steven J. Young et al., titled “APPARATUS FOR MONITORING VITAL SIGNS,” the entire contents of which is incorporated herein by reference.

For example, the pressure transducer 46 can be used to monitor the air pressure in the chambers 14A and 14B of the bed 12. If the user on the bed 12 is not moving, the air pressure changes in the air chamber 14A or 14B can be relatively minimal, and can be attributable to respiration and heartbeat. When the user on the bed 12 is moving, however, the air pressure in the mattress may fluctuate by a much larger amount. Thus, the pressure signals generated by the pressure transducer 46 and received by the processor 36 can be filtered and indicated as corresponding to motion, heartbeat, or respiration.

In some implementations, rather than performing the data analysis in the control box 24 with the processor 36, a digital signal processor (DSP) can be provided to analyze the data collected by the pressure transducer 46. Alternatively, the data collected by the pressure transducer 46 could be sent to a cloud-based computing system for remote analysis.

In some implementations, the example air bed system 10 further includes a temperature controller configured to increase, decrease, or maintain the temperature of a user. For example, a pad can be placed on top of or be part of the bed 12, or can be placed on top of or be part of one or both of the chambers 14A and 14B. Air can be pushed through the pad and vented to cool off a user of the bed. Conversely, the pad may include a heating element that can be used to keep the user warm. In some implementations, the temperature controller can receive temperature readings from the pad. In some implementations, separate pads are used for the different sides of the bed 12 (e.g., corresponding to the locations of the chambers 14A and 14B) to provide for differing temperature control for the different sides of the bed.

In some implementations, the user of the system 10 can use an input device, such as the remote control 22, to input a desired temperature for the surface of the bed 12 (or for a portion of the surface of the bed 12). The desired temperature can be encapsulated in a command data structure that includes the desired temperature as well as identifies the temperature controller as the desired component to be controlled. The command data structure may then be transmitted via Bluetooth or another suitable communication protocol to the processor 36. In various examples, the command data structure is encrypted before being transmitted. The temperature controller may then configure its elements to increase or decrease the temperature of the pad depending on the temperature input into remote control 22 by the user.

In some implementations, data can be transmitted from a component back to the processor 36 or to one or more display devices, such as the display 26. For example, the current temperature as determined by a sensor element of temperature controller, the pressure of the bed, the current position of the foundation or other information can be transmitted to control box 24. The control box 24 may then transmit the received information to remote control 22 where it can be displayed to the user (e.g., on the display 26).

In some implementations, the example air bed system 10 further includes an adjustable foundation and an articulation controller configured to adjust the position of a bed (e.g., the bed 12) by adjusting the adjustable foundation that supports the bed. For example, the articulation controller can adjust the bed 12 from a flat position to a position in which a head portion of a mattress of the bed is inclined upward (e.g., to facilitate a user sitting up in bed and/or watching television). In some implementations, the bed 12 includes multiple separately articulable sections. For example, portions of the bed corresponding to the locations of the chambers 14A and 14B can be articulated independently from each other, to allow one person positioned on the bed 12 surface to rest in a first position (e.g., a flat position) while a second person rests in a second position (e.g., an reclining position with the head raised at an angle from the waist). In some implementations, separate positions can be set for two different beds (e.g., two twin beds placed next to each other). The foundation of the bed 12 may include more than one zone that can be independently adjusted. The articulation controller may also be configured to provide different levels of massage to one or more users on the bed 12.

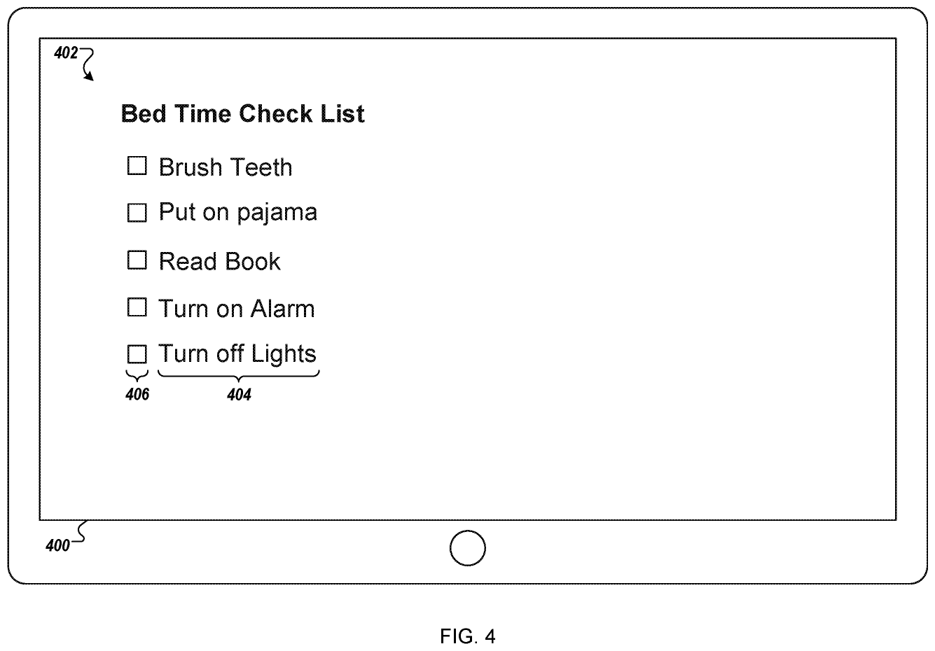

FIG. 3 shows an example environment 300 including a bed 302 in communication with devices located in and around a home. In the example shown, the bed 302 includes pump 304 for controlling air pressure within two air chambers 306a and 306b (as described above with respect to the air chambers 14A-14B). The pump 304 additionally includes circuitry for controlling inflation and deflation functionality performed by the pump 304. The circuitry is further programmed to detect fluctuations in air pressure of the air chambers 306a-b and used the detected fluctuations in air pressure to identify bed presence of a user 308, sleep state of the user 308, movement of the user 308, and biometric signals of the user 308 such as heart rate and respiration rate. In the example shown, the pump 304 is located within a support structure of the bed 302 and the control circuitry for controlling the pump 304 is integrated with the pump 304. In some implementations, the control circuitry is physically separate from the pump 304 and is in wireless or wired communication with the pump 304. In some implementations, the pump 304 and/or control circuitry are located outside of the bed 302. In some implementations, various control functions can be performed by systems located in different physical locations. For example, circuitry for controlling actions of the pump 304 can be located within a pump casing of the pump 304 while control circuitry for performing other functions associated with the bed 302 can be located in another portion of the bed 302, or external to the bed 302. As another example, control circuitry located within the pump 304 can communicate with control circuitry at a remote location through a LAN or WAN (e.g., the internet). As yet another example, the control circuitry can be included in the control box 24 of FIGS. 1 and 2.

In some implementations, one or more devices other than, or in addition to, the pump 304 and control circuitry can be utilized to identify user bed presence, sleep state, movement, and biometric signals. For example, the bed 302 can include a second pump in addition to the pump 304, with each of the two pumps connected to a respective one of the air chambers 306a-b. For example, the pump 304 can be in fluid communication with the air chamber 306b to control inflation and deflation of the air chamber 306a as well as detect user signals for a user located over the air chamber 306b such as bed presence, sleep state, movement, and biometric signals while the second pump is in fluid communication with the air chamber 306a to control inflation and deflation of the air chamber 306a as well as detect user signals for a user located over the air chamber 306a.

As another example, the bed 302 can include one or more pressure sensitive pads or surface portions that are operable to detect movement, including user presence, user motion, respiration, and heart rate. For example, a first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located during sleep. The movement detected by the one or more pressure sensitive pads or surface portions can be used by control circuitry to identify user sleep state, bed presence, or biometric signals.

In some implementations, information detected by the bed (e.g., motion information) is processed by control circuitry (e.g., control circuitry integrated with the pump 304) and provided to one or more user devices such as a user device 310 for presentation to the user 308 or to other users. In the example depicted in FIG. 3, the user device 310 is a tablet device; however, in some implementations, the user device 310 can be a personal computer, a smart phone, a smart television (e.g., a television 312), or other user device capable of wired or wireless communication with the control circuitry. The user device 310 can be in communication with control circuitry of the bed 302 through a network or through direct point-to-point communication. For example, the control circuitry can be connected to a LAN (e.g., through a WiFi router) and communicate with the user device 310 through the LAN. As another example, the control circuitry and the user device 310 can both connect to the Internet and communicate through the Internet. For example, the control circuitry can connect to the Internet through a WiFi router and the user device 310 can connect to the Internet through communication with a cellular communication system. As another example, the control circuitry can communicate directly with the user device 310 through a wireless communication protocol such as Bluetooth. As yet another example, the control circuitry can communicate with the user device 310 through a wireless communication protocol such as ZigBee, Z-Wave, or another wireless communication protocol suitable for the application. As another example, the control circuitry can communicate with the user device 310 through a wired connection such as, for example, a USB connector or another wired connection suitable for the application.

The user device 310 can display a variety of information and statistics related to sleep for the user 308 or user interaction with the bed 302 by the user 308. For example, a user interface displayed by the user device 310 can present information including amount of sleep for the user 308 over a period of time (e.g., a single evening, a week, a month, etc.) amount of deep sleep, ratio of deep sleep to restless sleep, time lapse between the user 308 getting into bed and the user 308 falling asleep, total amount of time spent in the bed 302 for a given period of time, heart rate for the user 308 over a period of time, respiration rate for the user 308 over a period of time, or other information related to user interaction with the bed 302 by the user 308 or one or more other users of the bed 302. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308. For example, the information presented on the user device 310 can evolve with the age of the user 308 such that different information is presented on the user device 310 as the user 308 ages as a child or an adult.

The user device 310 can also be used as an interface for the control circuitry of the bed 302 to allow the user 308 to enter information. The information entered by the user 308 can be used by the control circuitry to provide better information to the user or to various control signals for controlling functions of the bed 302 or other devices. For example, the user can enter information such as weight, height, and age and the control circuitry can use this information to provide the user 308 with a comparison of the user's tracked sleep information to sleep information of other people having similar weights, heights, and/or ages as the user 308. As another example, the user 308 can use the user device 310 as an interface for controlling air pressure of the air chambers 306a and 306b, for controlling various recline or incline positions of the bed 302, for controlling temperature of one or more surface temperature control devices of the bed 302, or for allowing the control circuitry to generate control signals for other devices (as described in greater detail below).

In some implementations, control circuitry of the bed 302 (e.g., control circuitry integrated into the pump 304) can communicate with other devices or systems in addition to or instead of the user device 310. For example, the control circuitry can communicate with the television 312, a lighting system 314, a thermostat 316, a security system 318, or other house hold devices such as an oven 322, a coffee maker 324, a lamp 326, and a nightlight 328. Other examples of devices and/or systems that the control circuitry can communicate with include a system for controlling window blinds 330, one or more devices for detecting or controlling the states of one or more doors 332 (such as detecting if a door is open, detecting if a door is locked, or automatically locking a door), and a system for controlling a garage door 320 (e.g., control circuitry integrated with a garage door opener for identifying an open or closed state of the garage door 320 and for causing the garage door opener to open or close the garage door 320). Communications between the control circuitry of the bed 302 and other devices can occur through a network (e.g., a LAN or the Internet) or as point-to-point communication (e.g., using Bluetooth, radio communication, or a wired connection). In some implementations, control circuitry of different beds 302 can communicate with different sets of devices. For example, a kid bed may not communicate with and/or control the same devices as an adult bed. In some embodiments, the bed 302 can evolve with the age of the user such that the control circuitry of the bed 302 communicates with different devices as a function of age of the user.

The control circuitry can receive information and inputs from other devices/systems and use the received information and inputs to control actions of the bed 302 or other devices. For example, the control circuitry can receive information from the thermostat 316 indicating a current environmental temperature for a house or room in which the bed 302 is located. The control circuitry can use the received information (along with other information) to determine if a temperature of all or a portion of the surface of the bed 302 should be raised or lowered. The control circuitry can then cause a heating or cooling mechanism of the bed 302 to raise or lower the temperature of the surface of the bed 302. For example, the user 308 can indicate a desired sleeping temperature of 74 degrees while a second user of the bed 302 indicates a desired sleeping temperature of 72 degrees. The thermostat 316 can indicate to the control circuitry that the current temperature of the bedroom is 72 degrees. The control circuitry can identify that the user 308 has indicated a desired sleeping temperature of 74 degrees, and send control signals to a heating pad located on the user 308's side of the bed to raise the temperature of the portion of the surface of the bed 302 where the user 308 is located to raise the temperature of the user 308's sleeping surface to the desired temperature.

The control circuitry can also generate control signals controlling other devices and propagate the control signals to the other devices. In some implementations, the control signals are generated based on information collected by the control circuitry, including information related to user interaction with the bed 302 by the user 308 and/or one or more other users. In some implementations, information collected from one or more other devices other than the bed 302 are used when generating the control signals. For example, information relating to environmental occurrences (e.g., environmental temperature, environmental noise level, and environmental light level), time of day, time of year, day of the week, or other information can be used when generating control signals for various devices in communication with the control circuitry of the bed 302. For example, information on the time of day can be combined with information relating to movement and bed presence of the user 308 to generate control signals for the lighting system 314. In some implementations, rather than or in addition to providing control signals for one or more other devices, the control circuitry can provide collected information (e.g., information related to user movement, bed presence, sleep state, or biometric signals for the user 308) to one or more other devices to allow the one or more other devices to utilize the collected information when generating control signals. For example, control circuitry of the bed 302 can provide information relating to user interactions with the bed 302 by the user 308 to a central controller (not shown) that can use the provided information to generate control signals for various devices, including the bed 302.

Still referring to FIG. 3, the control circuitry of the bed 302 can generate control signals for controlling actions of other devices, and transmit the control signals to the other devices in response to information collected by the control circuitry, including bed presence of the user 308, sleep state of the user 308, and other factors. For example, control circuitry integrated with the pump 304 can detect an increase in pressure in the air chamber 306b and use this detected increase in air pressure to determine that the user 308 is present on the bed 302. In some implementations, the control circuitry can identify a heart rate or respiratory rate for the user 308 to identify that the increase in pressure is due to a person sitting, laying, or otherwise resting on the bed 302 rather than an inanimate object (such as a suitcase) having been placed on the bed 302. In some implementations, the information indicating user bed presence is combined with other information to identify a current or future likely state for the user 308. For example, a detected user bed presence at 11:00 am may indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry detects that the user 308 has left the bed 302 at 6:30 am (e.g., indicating that the user 308 has woken up for the day), and then later detects user bed presence of the user 308 at 7:30 am, the control circuitry can use this information that the newly detected user bed presence is likely temporary (e.g., while the user 308 ties her shoes before heading to work) rather than an indication that the user 308 is intending to stay on the bed 302 for an extended period.

In some implementations, the control circuitry is able to use collected information (including information related to user interaction with the bed 302 by the user 308, as well as environmental information, time information, and input received from the user) to identify use patterns for the user 308. For example, the control circuitry can use information indicating bed presence and sleep states for the user 308 collected over a period of time to identify a sleep pattern for the user. For example, the control circuitry can identify that the user 308 generally goes to bed between 9:30 pm and generally falls asleep between 10:00 pm and 11:00 pm, and generally wakes up between 6:30 am and 6:45 am based on information indicating user presence and biometrics for the user 308 collected over a week. The control circuitry can use identified patterns for a user to better process and identify user interactions with the bed 302 by the user 308. For example, given the above example user bed presence, sleep, and wake patterns for the user 308, if the user 308 is detected as being on the bed at 3:00 pm, the control circuitry can determine that the user's presence on the bed is only temporary, and use this determination to generate different control signals than would be generated if the control circuitry determined that the user 308 was in bed for the evening. As another example, if the control circuitry detects that the user 308 has gotten out of bed at 3:00 am, the control circuitry can use identified patterns for the user 308 to determine that the user has only gotten up temporarily (for example, to use the rest room, or get a glass of water) and is not up for the day. By contrast, if the control circuitry identifies that the user 308 has gotten out of the bed 302 at 6:40 am, the control circuitry can determine that the user is up for the day and generate a different set of control signals than those that would be generated if it were determined that the user 308 were only getting out of bed temporarily (as would be the case when the user 308 gets out of the bed 302 at 3:00 am). For other users 308, getting out of the bed 302 at 3:00 am may be the normal wake-up time, which the control circuitry can learn and respond to accordingly.

As described above, the control circuitry for the bed 302 can generate control signals for control functions of various other devices. The control signals can be generated, at least in part, based on detected interactions by the user 308 with the bed 302, as well as other information including time, date, temperature, etc. For example, the control circuitry can communicate with the television 312, receive information from the television 312, and generate control signals for controlling functions of the television 312. For example, the control circuitry can receive an indication from the television 312 that the television 312 is currently on. If the television 312 is located in a different room from the bed 302, the control circuitry can generate a control signal to turn the television 312 off upon making a determination that the user 308 has gone to bed for the evening. For example, if bed presence of the user 308 in the bed 302 is detected during a particular time range (e.g., between 8:00 pm and 7:00 am) and persists for longer than a threshold period of time (e.g., 10 minutes) the control circuitry can use this information to determine that the user 308 is in bed for the evening. If the television 312 is on (as indicated by communications received by the control circuitry of the bed 302 from the television 312) the control circuitry can generate a control signal to turn the television 312 off. The control signals can then be transmitted to the television (e.g., through a directed communication link between the television 312 and the control circuitry or through a network). As another example, rather than turning off the television 312 in response to detection of user bed presence, the control circuitry can generate a control signal that causes the volume of the television 312 to be lowered by a pre-specified amount.

As another example, upon detecting that the user 308 has left the bed 302 during a specified time range (e.g., between 6:00 am and 8:00 am) the control circuitry can generate control signals to cause the television 312 to turn on and tune to a pre-specified channel (e.g., the user 308 has indicated a preference for watching the morning news upon getting out of bed in the morning). The control circuitry can generate the control signal and transmit the signal to the television 312 to cause the television 312 to turn on and tune to the desired station (which could be stored at the control circuitry, the television 312, or another location). As another example, upon detecting that the user 308 has gotten up for the day, the control circuitry can generate and transmit control signals to cause the television 312 to turn on and begin playing a previously recorded program from a digital video recorder (DVR) in communication with the television 312.

As another example, if the television 312 is in the same room as the bed 302, the control circuitry does not cause the television 312 to turn off in response to detection of user bed presence. Rather, the control circuitry can generate and transmit control signals to cause the television 312 to turn off in response to determining that the user 308 is asleep. For example, the control circuitry can monitor biometric signals of the user 308 (e.g., motion, heart rate, respiration rate) to determine that the user 308 has fallen asleep. Upon detecting that the user 308 is sleeping, the control circuitry generates and transmits a control signal to turn the television 312 off. As another example, the control circuitry can generate the control signal to turn off the television 312 after a threshold period of time after the user 308 has fallen asleep (e.g., 10 minutes after the user has fallen asleep). As another example, the control circuitry generates control signals to lower the volume of the television 312 after determining that the user 308 is asleep. As yet another example, the control circuitry generates and transmits a control signal to cause the television to gradually lower in volume over a period of time and then turn off in response to determining that the user 308 is asleep.

In some implementations, the control circuitry can similarly interact with other media devices, such as computers, tablets, smart phones, stereo systems, etc. For example, upon detecting that the user 308 is asleep, the control circuitry can generate and transmit a control signal to the user device 310 to cause the user device 310 to turn off, or turn down the volume on a video or audio file being played by the user device 310.

The control circuitry can additionally communicate with the lighting system 314, receive information from the lighting system 314, and generate control signals for controlling functions of the lighting system 314. For example, upon detecting user bed presence in the bed 302 during a certain time frame (e.g., between 8:00 pm and 7:00 am) that lasts for longer than a threshold period of time (e.g., 10 minutes) the control circuitry of the bed 302 can determine that the user 308 is in bed for the evening. In response to this determination, the control circuitry can generate control signals to cause lights in one or more rooms other than the room in which the bed 302 is located to switch off. The control signals can then be transmitted to the lighting system 314 and executed by the lighting system 314 to cause the lights in the indicated rooms to shut off. For example, the control circuitry can generate and transmit control signals to turn off lights in all common rooms, but not in other bedrooms. As another example, the control signals generated by the control circuitry can indicate that lights in all rooms other than the room in which the bed 302 is located are to be turned off, while one or more lights located outside of the house containing the bed 302 are to be turned on, in response to determining that the user 308 is in bed for the evening. Additionally, the control circuitry can generate and transmit control signals to cause the nightlight 328 to turn on in response to determining user 308 bed presence or whether the user 308 is asleep. As another example, the control circuitry can generate first control signals for turning off a first set of lights (e.g., lights in common rooms) in response to detecting user bed presence, and second control signals for turning off a second set of lights (e.g., lights in the room in which the bed 302 is located) in response to detecting that the user 308 is asleep.

In some implementations, in response to determining that the user 308 is in bed for the evening, the control circuitry of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep, and therefore is logically implemented when the control circuitry has determined that the user 308 is in bed for the evening.

The control circuitry can also be configured to implement a sunrise lighting scheme when the user 308 wakes up in the morning. The control circuitry can determine that the user 308 is awake for the day, for example, by detecting that the user 308 has gotten off of the bed 302 (i.e., is no longer present on the bed 302) during a specified time frame (e.g., between 6:00 am and 9:00 am). As another example, the control circuitry can monitor movement, heart rate, respiratory rate, or other biometric signals of the user 308 to determine that the user 308 is awake even though the user 308 has not gotten out of bed. If the control circuitry detects that the user is awake during a specified time frame, the control circuitry can determine that the user 308 is awake for the day. The specified time frame can be, for example, based on previously recorded user bed presence information collected over a period of time (e.g., two weeks) that indicates that the user 308 usually wakes up for the day between 6:30 am and 7:30 am. In response to the control circuitry determining that the user 308 is awake, the control circuitry can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. For example, the sunrise lighting scheme can include lighting the bedroom with blue light to gently assist the user 308 in waking up and becoming active.

In some implementations, the control circuitry can generate different control signals for controlling actions of the lighting system 314 depending on a time of day that user interactions with the bed 302 are detected. For example, the control circuitry can use historical user interaction information for interactions between the user 308 and the bed 302 to determine that the user 308 usually falls asleep between 10:00 pm and 11:00 pm and usually wakes up between 6:30 am and 7:30 am on weekdays. The control circuitry can use this information to generate a first set of control signals for controlling the lighting system 314 if the user 308 is detected as getting out of bed at 3:00 am and to generate a second set of control signals for controlling the lighting system 314 if the user 308 is detected as getting out of bed after 6:30 am. For example, if the user 308 gets out of bed prior to 6:30 am, the control circuitry can turn on lights that guide the user 308's route to a restroom. As another example, if the user 308 gets out of bed prior to 6:30 am, the control circuitry can turn on lights that guide the user 308's route to the kitchen (which can include, for example, turning on the nightlight 328, turning on under bed lighting, or turning on the lamp 326).

As another example, if the user 308 gets out of bed after 6:30 am, the control circuitry can generate control signals to cause the lighting system 314 to initiate a sunrise lighting scheme, or to turn on one or more lights in the bedroom and/or other rooms. In some implementations, if the user 308 is detected as getting out of bed prior to a specified morning rise time for the user 308, the control circuitry causes the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (i.e., prior to normal rise time for the user 308) can prevent other occupants of the house from being woken by the lights while still allowing the user 308 to see in order to reach the restroom, kitchen, or another destination within the house.

The historical user interaction information for interactions between the user 308 and the bed 302 can be used to identify user sleep and awake time frames. For example, user bed presence times and sleep times can be determined for a set period of time (e.g., two weeks, a month, etc.). The control circuitry can then identify a typical time frame in which the user 308 goes to bed, a typical time frame for when the user 308 falls asleep, and a typical time frame for when the user 308 wakes up (and in some cases, different time frames for when the user 308 wakes up and when the user 308 actually gets out of bed). In some implementations, buffer time can be added to these time frames. For example, if the user is identified as typically going to bed between 10:00 pm and a buffer of a half hour in each direction can be added to the time frame such that any detection of the user getting onto the bed between 9:30 pm and 11:00 pm is interpreted as the user 308 going to bed for the evening. As another example, detection of bed presence of the user starting from a half hour before the earliest typical time that the user 308 goes to bed extending until the typical wake up time (e.g., 6:30 am) for the user can be interpreted as the user going to bed for the evening. For example, if the user typically goes to bed between 10:00 pm and 10:30 pm, if the user's bed presence is sensed at 12:30 am one night, that can be interpreted as the user getting into bed for the evening even though this is outside of the user's typical time frame for going to bed because it has occurred prior to the user's normal wake up time. In some implementations, different time frames are identified for different times of the year (e.g., earlier bed time during winter vs. summer) or at different times of the week (e.g., user wakes up earlier on weekdays than on weekends).

The control circuitry can additionally communicate with the thermostat 316, receive information from the thermostat 316, and generate control signals for controlling functions of the thermostat 316. For example, the user 308 can indicate user preferences for different temperatures at different times, depending on the sleep state or bed presence of the user 308. For example, the user 308 may prefer an environmental temperature of 72 degrees when out of bed, 70 degrees when in bed but awake, and 68 degrees when sleeping. The control circuitry of the bed 302 can detect bed presence of the user 308 in the evening and determine that the user 308 is in bed for the night. In response to this determination, the control circuitry can generate control signals to cause the thermostat to change the temperature to 70 degrees. The control circuitry can then transmit the control signals to the thermostat 316. Upon detecting that the user 308 is asleep, the control circuitry can generate and transmit control signals to cause the thermostat 316 to change the temperature to 68. The next morning, upon determining that the user is awake for the day (e.g., the user 308 gets out of bed after 6:30 am) the control circuitry can generate and transmit control circuitry to cause the thermostat to change the temperature to 72 degrees.

In some implementations, the control circuitry can similarly generate control signals to cause one or more heating or cooling elements on the surface of the bed 302 to change temperature at various times, either in response to user interaction with the bed 302 or at various pre-programmed times. For example, the control circuitry can activate a heating element to raise the temperature of one side of the surface of the bed 302 to 73 degrees when it is detected that the user 308 has fallen asleep. As another example, upon determining that the user 308 is up for the day, the control circuitry can turn off a heating or cooling element. As yet another example, the user 308 can pre-program various times at which the temperature at the surface of the bed should be raised or lowered. For example, the user can program the bed 302 to raise the surface temperature to 76 degrees at 10:00 pm, and lower the surface temperature to 68 degrees at 11:30 pm.

In some implementations, in response to detecting user bed presence of the user 308 and/or that the user 308 is asleep, the control circuitry can cause the thermostat 316 to change the temperature in different rooms to different values. For example, in response to determining that the user 308 is in bed for the evening, the control circuitry can generate and transmit control signals to cause the thermostat 316 to set the temperature in one or more bedrooms of the house to 72 degrees and set the temperature in other rooms to 67 degrees.

The control circuitry can also receive temperature information from the thermostat 316 and use this temperature information to control functions of the bed 302 or other devices. For example, as discussed above, the control circuitry can adjust temperatures of heating elements included in the bed 302 in response to temperature information received from the thermostat 316.

In some implementations, the control circuitry can generate and transmit control signals for controlling other temperature control systems. For example, in response to determining that the user 308 is awake for the day, the control circuitry can generate and transmit control signals for causing floor heating elements to activate. For example, the control circuitry can cause a floor heating system for a master bedroom to turn on in response to determining that the user 308 is awake for the day.

The control circuitry can additionally communicate with the security system 318, receive information from the security system 318, and generate control signals for controlling functions of the security system 318. For example, in response to detecting that the user 308 in is bed for the evening, the control circuitry can generate control signals to cause the security system to engage security functions. The control circuitry can then transmit the control signals to the security system 318 to cause the security system 318 to engage. As another example, the control circuitry can generate and transmit control signals to cause the security system 318 to disable in response to determining that the user 308 is awake for the day (e.g., user 308 is no longer present in the bed 302 after 6:00 am). In some implementations, the control circuitry can generate and transmit a first set of control signals to cause the security system 318 to engage a first set of security features in response to detecting user bed presence of the user 308, and can generate and transmit a second set of control signals to cause the security system 318 to engage a second set of security features in response to detecting that the user 308 has fallen asleep.

In some implementations, the control circuitry can receive alerts from the security system 318 and indicate the alert to the user 308. For example, the control circuitry can detect that the user 308 is in bed for the evening and in response, generate and transmit control signals to cause the security system 318 to engage. The security system can then detect a security breach (e.g., someone has opened the door 332 without entering the security code, or someone has opened a window when the security system 318 is engaged). The security system 318 can communicate the security breach to the control circuitry of the bed 302. In response to receiving the communication from the security system 318, the control circuitry can generate control signals to alert the user 308 to the security breach. For example, the control circuitry can cause the bed 302 to vibrate. As another example, the control circuitry can cause portions of the bed 302 to articulate (e.g., cause the head section to raise or lower) in order to wake the user 308 and alert the user to the security breach. As another example, the control circuitry can generate and transmit control signals to cause the lamp 326 to flash on and off at regular intervals to alert the user 308 to the security breach. As yet another example, the control circuitry can generate an audible alarm from hardware (e.g., a speaker, a bell) incorporated in the bed. As another example, the control circuitry can alert the user 308 of one bed 302 regarding a security breach in a bedroom of another bed, such as an open window in a kid's bedroom.

The control circuitry can additionally generate and transmit control signals for controlling the garage door 320 and receive information indicating a state of the garage door 320 (i.e., open or closed). For example, in response to determining that the user 308 is in bed for the evening, the control circuitry can generate and transmit a request to a garage door opener or another device capable of sensing if the garage door 320 is open. The request can request information on the current state of the garage door 320. If the control circuitry receives a response (e.g., from the garage door opener) indicating that the garage door 320 is open, the control circuitry can either notify the user 308 that the garage door is open, or generate a control signal to cause the garage door opener to close the garage door 320. For example, the control circuitry can send a message to the user device 310 indicating that the garage door is open. As another example, the control circuitry can cause the bed 302 to vibrate. As yet another example, the control circuitry can generate and transmit a control signal to cause the lighting system 314 to cause one or more lights in the bedroom to flash to alert the user 308 to check the user device 310 for an alert (in this example, an alert regarding the garage door 320 being open). Alternatively, or additionally, the control circuitry can generate and transmit control signals to cause the garage door opener to close the garage door 320 in response to identifying that the user 308 is in bed for the evening and that the garage door 320 is open. In some implementations, control signals can vary depend on the age of the user 308.

The control circuitry can similarly send and receive communications for controlling or receiving state information associated with the door 332 or the oven 322. For example, upon detecting that the user 308 is in bed for the evening, the control circuitry can generate and transmit a request to a device or system for detecting a state of the door 332. Information returned in response to the request can indicate various states for the door 332 such as open, closed but unlocked, or closed and locked. If the door 332 is open or closed but unlocked, the control circuitry can alert the user 308 to the state of the door, such as in a manner described above with reference to the garage door 320. Alternatively, or in addition to alerting the user 308, the control circuitry can generate and transmit control signals to cause the door 332 to lock, or to close and lock. If the door 332 is closed and locked, the control circuitry can determine that no further action is needed.

Similarly, upon detecting that the user 308 is in bed for the evening, the control circuitry can generate and transmit a request to the oven 322 to request a state of the oven 322 (e.g., on or off). If the oven 322 is on, the control circuitry can alert the user 308 and/or generate and transmit control signals to cause the oven 322 to turn off. If the oven is already off, the control circuitry can determine that no further action is necessary. In some implementations, different alerts can be generated for different events. For example, the control circuitry can cause the lamp 326 (or one or more other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user of that bed has gotten up (e.g., that a child of the user 308 has gotten out of bed in the middle of the night as sensed by a sensor in the bed 302 of the child). Other examples of alerts that can be processed by the control circuitry of the bed 302 and communicated to the user include a smoke detector detecting smoke (and communicating this detection of smoke to the control circuitry), a carbon monoxide tester detecting carbon monoxide, a heater malfunctioning, or an alert from any other device capable of communicating with the control circuitry and detecting an occurrence that should be brought to the user 308's attention.

The control circuitry can also communicate with a system or device for controlling a state of the window blinds 330. For example, in response to determining that the user 308 is in bed for the evening, the control circuitry can generate and transmit control signals to cause the window blinds 330 to close. As another example, in response to determining that the user 308 is up for the day (e.g., user has gotten out of bed after 6:30 am) the control circuitry can generate and transmit control signals to cause the window blinds 330 to open. By contrast, if the user 308 gets out of bed prior to a normal rise time for the user 308, the control circuitry can determine that the user 308 is not awake for the day and does not generate control signals for causing the window blinds 330 to open. As yet another example, the control circuitry can generate and transmit control signals that cause a first set of blinds to close in response to detecting user bed presence of the user 308 and a second set of blinds to close in response to detecting that the user 308 is asleep.

The control circuitry can generate and transmit control signals for controlling functions of other household devices in response to detecting user interactions with the bed 302. For example, in response to determining that the user 308 is awake for the day, the control circuitry can generate and transmit control signals to the coffee maker 324 to cause the coffee maker 324 to begin brewing coffee. As another example, the control circuitry can generate and transmit control signals to the oven 322 to cause the oven to begin preheating (for users that like fresh baked bread in the morning). As another example, the control circuitry can use information indicating that the user 308 is awake for the day along with information indicating that the time of year is currently winter and/or that the outside temperature is below a threshold value to generate and transmit control signals to cause a car engine block heater to turn on.

As another example, the control circuitry can generate and transmit control signals to cause one or more devices to enter a sleep mode in response to detecting user bed presence of the user 308, or in response to detecting that the user 308 is asleep. For example, the control circuitry can generate control signals to cause a mobile phone of the user 308 to switch into sleep mode. The control circuitry can then transmit the control signals to the mobile phone. Later, upon determining that the user 308 is up for the day, the control circuitry can generate and transmit control signals to cause the mobile phone to switch out of sleep mode.

In some implementations, the control circuitry can communicate with one or more noise control devices. For example, upon determining that the user 308 is in bed for the evening, or that the user 308 is asleep, the control circuitry can generate and transmit control signals to cause one or more noise cancelation devices to activate. The noise cancelation devices can, for example, be included as part of the bed 302 or located in the bedroom with the bed 302. As another example, upon determining that the user 308 is in bed for the evening or that the user 308 is asleep, the control circuitry can generate and transmit control signals to turn the volume on, off, up, or down, for one or more sound generating devices, such as a stereo system radio, computer, tablet, etc.

Additionally, functions of the bed 302 are controlled by the control circuitry in response to user interactions with the bed 302. For example, the bed 302 can include an adjustable foundation and an articulation controller configured to adjust the position of one or more portions of the bed 302 by adjusting the adjustable foundation that supports the bed. For example, the articulation controller can adjust the bed 302 from a flat position to a position in which a head portion of a mattress of the bed 302 is inclined upward (e.g., to facilitate a user sitting up in bed and/or watching television). In some implementations, the bed 302 includes multiple separately articulable sections. For example, portions of the bed corresponding to the locations of the chambers 306a and 306b can be articulated independently from each other, to allow one person positioned on the bed 302 surface to rest in a first position (e.g., a flat position) while a second person rests in a second position (e.g., a reclining position with the head raised at an angle from the waist). In some implementations, separate positions can be set for two different beds (e.g., two twin beds placed next to each other). The foundation of the bed 302 may include more than one zone that can be independently adjusted. The articulation controller may also be configured to provide different levels of massage to one or more users on the bed 302 or to cause the bed to vibrate to communicate alerts to the user 308 as described above.

The control circuitry can adjust positions (e.g., incline and decline positions for the user 308 and/or an additional user of the bed 302) in response to user interactions with the bed 302. For example, the control circuitry can cause the articulation controller to adjust the bed 302 to a first recline position for the user 308 in response to sensing user bed presence for the user 308. The control circuitry can cause the articulation controller to adjust the bed 302 to a second recline position (e.g., a less reclined, or flat position) in response to determining that the user 308 is asleep. As another example, the control circuitry can receive a communication from the television 312 indicating that the user 308 has turned off the television 312, and in response the control circuitry can cause the articulation controller to adjust the position of the bed 302 to a preferred user sleeping position (e.g., due to the user turning off the television 312 while the user 308 is in bed indicating that the user 308 wishes to go to sleep).

In some implementations, the control circuitry can control the articulation controller so as to wake up one user of the bed 302 without waking another user of the bed 302. For example, the user 308 and a second user of the bed 302 can each set distinct wakeup times (e.g., 6:30 am and 7:15 am respectively). When the wakeup time for the user 308 is reached, the control circuitry can cause the articulation controller to vibrate or change the position of only a side of the bed on which the user 308 is located to wake the user 308 without disturbing the second user. When the wakeup time for the second user is reached, the control circuitry can cause the articulation controller to vibrate or change the position of only the side of the bed on which the second user is located. Alternatively, when the second wakeup time occurs, the control circuitry can utilize other methods (such as audio alarms, or turning on of lights) to wake the second user since the user 308 is already awake and therefore will not be disturbed when the control circuitry attempts to wake the second user.

Still referring to FIG. 3, the control circuitry for the bed 302 can utilize information for interactions with the bed 302 by multiple users to generate control signals for controlling functions of various other devices. For example, the control circuitry can wait to generate control signals for, for example, engaging the security system 318, or instructing the lighting system 314 to turn off lights in various rooms until both the user 308 and a second user are detected as being present on the bed 302. As another example, the control circuitry can generate a first set of control signals to cause the lighting system 314 to turn off a first set of lights upon detecting bed presence of the user 308 and generate a second set of control signals for turning off a second set of lights in response to detecting bed presence of a second user. As another example, the control circuitry can wait until it has been determined that both the user 308 and a second user are awake for the day before generating control signals to open the window blinds 330. As yet another example, in response to determining that the user 308 has left the bed and is awake for the day, but that a second user is still sleeping, the control circuitry can generate and transmit a first set of control signals to cause the coffee maker 324 to begin brewing coffee, to cause the security system 318 to deactivate, to turn on the lamp 326, to turn off the nightlight 328, to cause the thermostat 316 to raise the temperature in one or more rooms to 72 degrees, and to open blinds (e.g., the window blinds 330) in rooms other than the bedroom. Later, in response to detecting that the second user is no longer present in the bed (or that the second user is awake) the control circuitry can generate and transmit a second set of control signals to, for example, cause the lighting system 314 to turn on one or more lights in the bedroom, to cause window blinds in the bedroom to open, and to turn on the television 312 to a pre-specified channel.

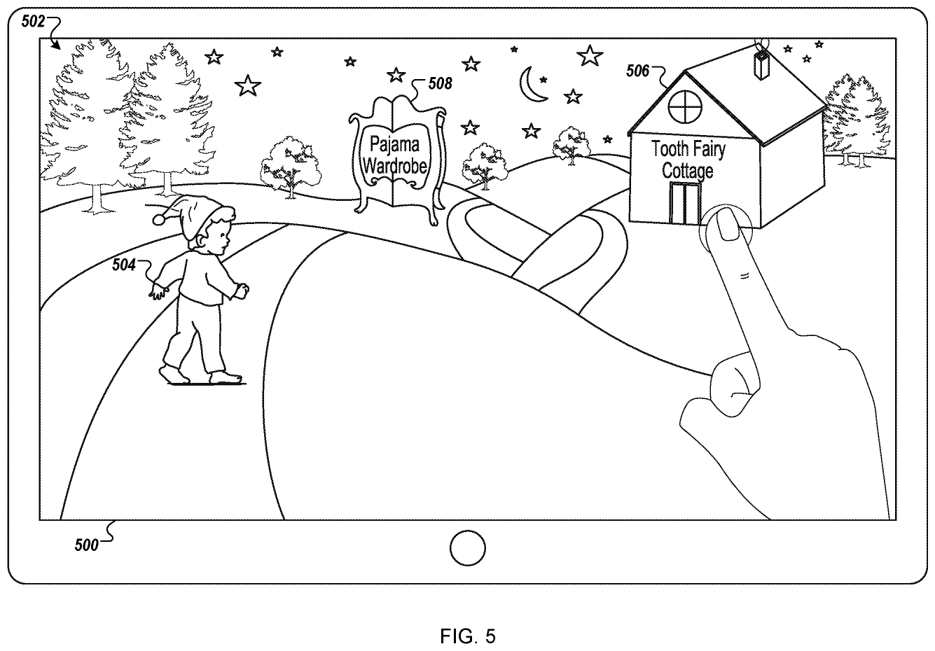

FIG. 4 is an example computer interface 400 showing a bedtime checklist application 402. This checklist application 402 may be, for example, displayed to a user at a programmed ‘bed time’ (e.g. 7:15 PM) with tasks selected by the user, their parent or guardian, or another party. This checklist application 402 can present an ordered list of tasks for the user to accomplish as part of a bed-time routine.

This computer interface 400 may be, for example, a touch screen interface that displays the checklist application 402 and receives input in the form of user finger presses on the screen. Example computing devices with such a computer interface 400 can include, but are not limited to, cellular phones, tablets, and laptop computers. Alternatively or additionally, the computer interface 400 may receive user input from peripheral devices such a keyboard, mouse, or stylus. In some implementations, the computer interface 400 can be integrated with the bed 302 (shown in FIG. 3). In some implementations, the computer interface 400 can be separate from the bed 302.

The checklist application can include an ordered list of actions 404, and for each action, an interactive interface element such as a checkbox 406. The user may be instructed, by onscreen instructions, by a parent or guardian, or otherwise, to complete each action 404 in the order that they are listed. When the user completes each action, the user can check the associated checkbox 406.

In the case of home use for a family with children, the actions 404 may be set by a parent or guardian that plans the child's bedtime routine. In other environments, the actions 404 may be set by other parties. For example, in an enterprise situation in which employee bedtime routes may be tracked (e.g., long-haul trucking, multi-day fishing voyages, summer camp counseling), the actions 404 may be set by an enterprise to ensure that job duties and safety measure are taken before the user goes to bed. The tasks 404 may also be users to communicate with caretakers. A user in assisted living or that receives caregiving may use the tasks 404 to communicate to their care giver that they are able and remember to perform all of their bedtime activities. For example, a son or daughter with an older parent living in an over-garage apartment may use the checklist application to monitor the parent's activity for safety purposes, while still affording the parent autonomy and privacy.

While the user is performing each action 404 of the checklist, the checklist application 402, or another application in communicating with the application 402, can monitor the input to the checklist application 402 and determine the state of the user. The state of the user can include the location of the user, the current action 404 to which the user is working, the state of the user's clothing (e.g., if they have changed into pajamas), and/or the state of the user's evening hygiene requirements (e.g., if they have brushed their teeth and/or washed their face).

In addition to the input to the checklist application 402, there are other inputs and outputs that may be used to track the state of the user. For example, the pump 304 can detect presence in the user's bed 302, and the lighting system 314 may be turned on or off to illuminate or darken the bedroom containing the bed 302. If the next unchecked action 404 is “Read a Book,” the lighting system 314 is turned on to illuminate the bedroom, and if the pump 304 detects a presence in the bed, the application 402 can determine that the user's state is reading a book in the bed 302.

The application 402 can also engage some outputs to facilitate an activity associated with the user's state. For example, the application 402 can determine that the user's current state is ‘turn out the lights.’ In some cases, the lighting system 314 may automatically turn off when the application 402 determines the user's state. Alternatively, the display 400 can temporarily remove the checklist application 402 and replace it with an interface to control the lighting system 314. In yet another alternative, the application 402 can turn on an under-bed lighting system (not shown) to facilitate the user turning off the lights via a light switch on a wall (not shown). In some examples, the checklist application 402 can be programmed to evolve with the age of the user, such that the actions 404 in the checklist application 402 vary depending on the age of the user. For example, “Read Book” could be added when the user turns a certain age (e.g. 6 years old) and “Turn on Alarm” could be added when the user turns a different, older age (e.g. 16 years old). In some examples, the checklist application 402 can evolve with the time of year, such that the actions 404 in the checklist application 402 vary depending on the time of year. For example, “Read Book” could be added during summer month and “Do Homework” could be added during fall, winter, and spring.

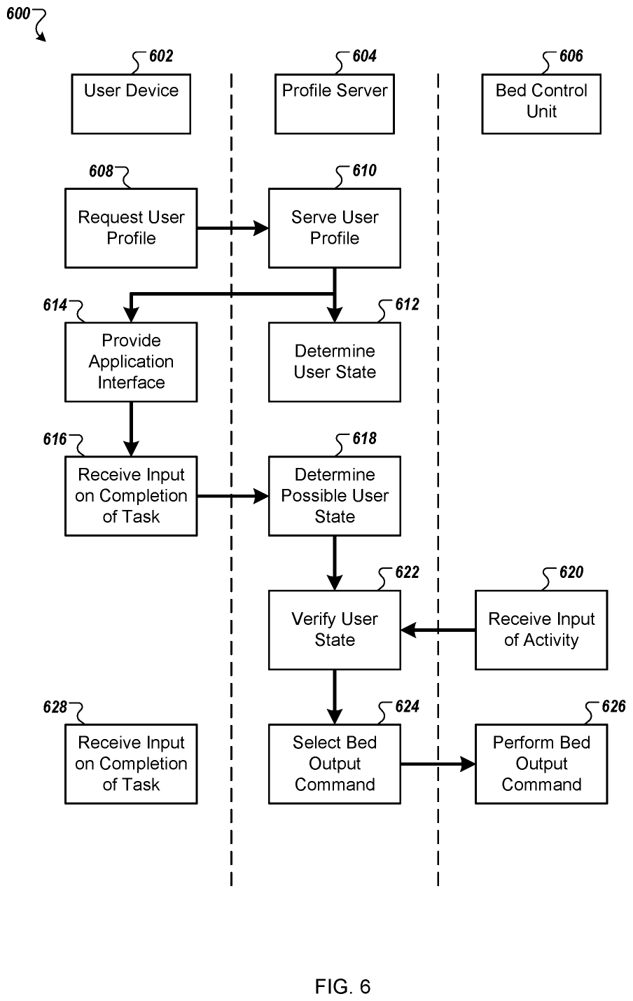

FIG. 5 is an example computer interface 500 showing a bedtime videogame application 502. This videogame application 502 may be, for example, displayed to a user at a programmed ‘bed time’ (e.g. 7:15 PM) with minigames selected by the user, their parent or guardian, or another party. This videogame application 502 can present an ordered list of tasks for the user to accomplish as part of a bed-time routine.

In this example, the user is represented by an avatar 504 walking down a lane from one minigame 506 to the next 508. The first minigame 506 is a visit to the Tooth Fairy Cottage. To complete this minigame 506, the user guides the avatar through a tooth-brushing minigame, followed by the user brushing their own teeth. Once complete, the user can move the avatar 506 to the next minigame 508, where the avatar 504 and the user change into their pajamas.

Similar to the application 402, the application 502 may track a user's state when they use the application 502. In this example, after the tooth brushing mini-game is completed, the application 502 can determine that the user is going to be in a pajama changing state. As described previously, additional inputs may also be used by the application 502 to track the user's state, and additional outputs may be engaged to assist the user in their current state.

Two types of applications that are capable of tracking user state have been described, but it will be appreciated that there are other applications capable of performing similar user tracking. In the two examples shown, a user is assisted in their bedtime routine with the assignment of a series of actions. These actions may have been selected by the user themselves, or by another person. For example, a parent or guardian of the user may select and order actions appropriate to the user's age, temperament, and the needs of the user's family (e.g., scheduling different users brushing their teeth at different times). The application may provide the parent or guardian with suggestions for these actions based on the user's age, history using the application, or other appropriate sources of knowledge.

The application may have multiple age or development appropriate interfaces. For example, the same application may display as the application 502 to a younger child and as the application 402 to an older child that has better reading skills but may not have the patience for the animations of the application 502. In some examples, the interface can be programmed to evolve with the user according to the age of the user to display age-appropriate interfaces automatically or as selected by the user.

In any case, some or all of the actions may be associated with external input devices and output devices in the application. When the application determines that the user is or may be in a state related to one action, the application can verify the user's state based on input from a related input device. For example, an illumination sensor may be set to detect illumination in the user's library or family room. An action in the application called “read a book in the family room” may indicate to the application that the user may be in the state of reading a book in the family room, and input from the illumination sensor may verify that the family room is occupied based on the lights in the family room being on and illuminating the room. In another example, an illumination sensor in the bedroom (integrated with the bed 302, for example) can verify whether the user has, in fact, turned off the lights as indicated on the bedtime checklist application 402. In another example, a bed presence sensor integrated with the bed 302 can verify if the user has, in fact, gone to bed, if going to bed is set as an action on the bedtime checklist application 402.

Similarly, some actions may be associated with output devices. An action ‘read a book in bed’ may be tied to an elevation bladder in the bed 302. When the user enters the state of “read a book in bed,” the elevation bladder may inflate, elevating the head of the bed for more comfortable reading. If the output device is associated with a delay (e.g., the bladder takes four minutes to inflate), the application can engage the output device an appropriate amount of time before the user is expected to enter the associated state.

The application can include other features not discussed with respect to FIGS. 4 and 5. For example, the application can be tied to a user account stored, for example, in the application or another application. This user account can include profile information (e.g., name, age, social media information) used exclusively by the application or in connection with other applications. For example, an application store profile may be used by this application and other video games that the user plays. Activity related to this application may result in transactions applied to the user's account, which may propagate to other applications associated with the user's account. In some examples, a child's profile can have access only to his or her own data while a parent or guardian's profile can have access to data of all profiles in a family group.

For example, the user may purchase and/or download a video game to their computer, phone, or other device through an application store. This video game may have in-game resources that are scarce, either available as rewards or in exchange for in-app purchases. The same user, using the same application store, may also purchase and/or download a bedtime application. If some goal in the bedtime application is met (e.g., staying in bed overnight, as detected by a bed presence sensor), the user's application store may be credited with currency or points applicable to the video game.

In another example, the bedtime application is tied to the user account of an electronic device such as a console gaming machine or tablet device. If the user meets some goal in the bedtime application (e.g., no detection of a bed wetting event by a sensor in the bed and/or staying in bed until morning without getting up), the user's account on the device may be unlocked the next day for a predetermined period of time. In another example, if the user meets some goal in the bedtime application (e.g., no detection of a bed wetting event by a sensor in the bed and/or staying in bed until morning without getting up), the user may be credited with an in-game reward or opportunity.

To this point, the example applications have been described with respect to tasks and states related to bedtime activities. However, a variety of other types of actives may be used instead. For example, instead of tracking a user's bedtime task, an application may be used to track a user's morning routine. Such a use may include tasks such as “get dressed,” “put away pajamas” “eat breakfast” “brush teeth,” and on morning with school scheduled “collect book bag.” As would be apparent, some tasks and states may be used for bedtime, morning, and other routines. A child's afternoon nap and evening bedtime may both include the task “use bathroom,” for example. The user interface in FIG. 5 is themed with an evening bedtime theme. However, other themes may be used for other types of tracking. For example, a morning theme with chirping birds and a rising sun may be used for an application tracking a user's state in the morning.

In some cases, an application can be used as part of an on-the-job routine. For example, an enterprise with on-call staff that may sleep while on-call may use an application to ensure that the staff is prepared to work after they awake. The enterprise may program the application to require the user to perform some tasks that demonstrate that they are awake and prepared. Such tasks may include dressing, washing hands, and taking a drink of water. Some tasks may be designed to test the user's attentiveness. For example, a task may require the user to perform a series of arithmetic questions or logic problem.

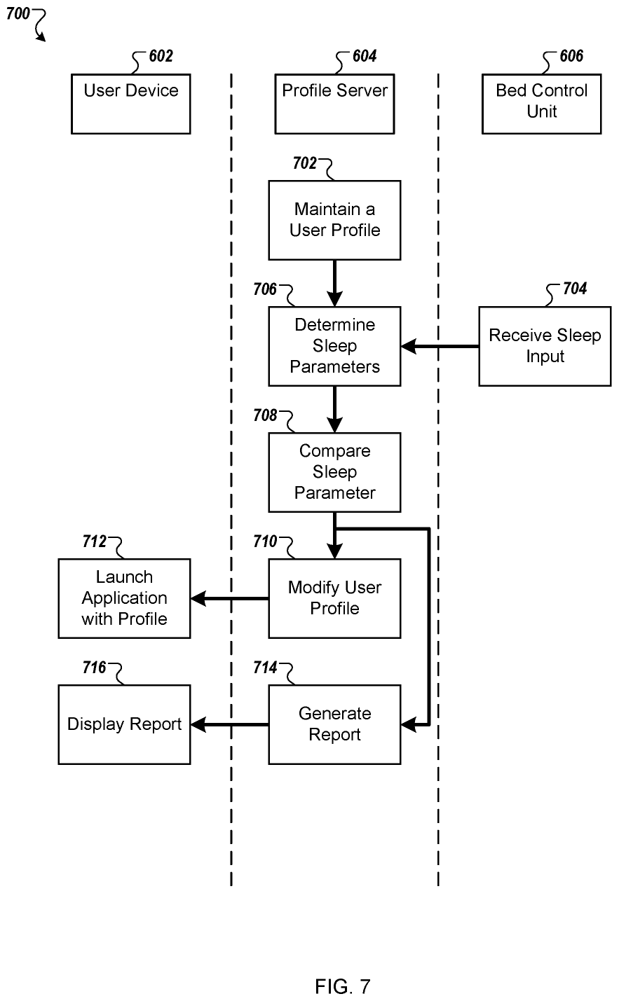

FIG. 6 is a swimlane diagram of an example process 600 for tracking a user's state and updating output devices. In the process 600, a computer system can track the state of a user and facilitate the user's bedtime routine by engaging appropriate output devices connected to the user's bed. The process 600 will be described with reference to a user device 602, a profile server 604, and a bed control unit 606. However, the process 600 and other similar processes may be performed by different devices, including but not limited to devices previously described in this document.

The user device 602 is any appropriate computing device or system that can display screen-based output to a user and receive input from the user. Example user devices 602 include but are not limited to desktop or laptop computers, mobile phones, tablet computers, video game consoles, and wearable or mounted computers. The profile server 604 is a computer server that is communicably coupled to the user device 602 and the bed control unit 606. The profile server 604 may be an independent computer device or a subsystem of the user device 602 and/or the bed control unit 606. The profile server 604 may be located in the same building as the user device, or it may be located in a different location or locations (e.g., distributed across multiple physical servers).

The bed control unit 606 may include computing hardware and software to control input and output devices associated with a user's bed and/or environment. For example, the bed control unit 606 may be one or more devices able to receive instructions from the user device 602 and/or the profile server 604, read input values from input devices (e.g., illumination sensors, pressure sensors, temperature sensors, bed moisture sensors) and drive output devices (e.g., bed inflation pumps, lighting systems, coffee makers, temperature controllers). Some or all of the input and output devices may be incorporated into the bed, or some or all may be coupled to the bed by a data plug, by a wireless data interface, or otherwise.

The user device 602 requests 608 a user profile. For example, the user may bring up a bedtime application, or at a predetermined time, an alert on the user device 602 may announce to the user that it is time to begin their bedtime routine. The bedtime application may call up the user's profile from the profile server 604, for example to receive any updates that have occurred since the last time that the bedtime application ran.

The profile server 604 serves the user profile 610. For example, the profile server 604 can pass the user profile, or data constructed based on the user profile, back to the user device 602. This data may take the form of any appropriate data structure including, but not limited to, an Extensible Markup Language (XML) data file, a JavaScript Object Notation (JSON) data object, or other well-known or custom data format.

The profile server 604 determines 612 a user state. For example, the user profile may include a series of tasks to be completed as part of the bedtime routine, or the user device 602 may store and report the series of tasks to the profile server 604. From this series of tasks, the profile server 604 may determine that the user is assigned to the first task, until that task is completed.

The user device 602 provides 614, to a user, a computer application interface. For example, the bedtime application may have a single user interface, or may select or construct the user interface based on information from the user profile. The computer application interface can include instructions to complete at least one bedtime task, along with other information or interface elements. For example, the interface may include a checklist or a game that the user plays while performing the task.

The user device 602 receives 616, from the user through the computer application interface, first input indicating that the user has completed a first task. For example, the user may check a box associated with the first task, or the user may complete a game that requires the task as part of completion. Upon completion of the task, the user device 602 may report the completion to the profile server 604.

The profile server 604 determines 618, based on the first input, that the user is possibly assigned to a second task. For example, if the first task is “brush teeth” and the second task is “turn off lights,” upon notification that the user has completed brushing their teeth, the profile server 604 can determine that the user is likely in the “turn off lights” task in the bedtime application.

The bed control unit 606 receives 620 second input, from the user, to a bed input device that is capable of detecting at least one physical phenomena associated with the bed. For example, when the user switches off their light, the bed control unit 606 may receive input from an illumination sensor that the illumination in the user's room around the user's bed has decreased. The bed control unit 606 can report this decrease to the profile server 604.

The profile server 604 determines 622, based on the first input and the second input, that the user is assigned to the second task. For example, the profile server 604 can access data associated with the “turn off lights” action and determine that this action is associated with a decrease in illumination. When the profile server 604 receives the notification of decrease in illumination from the bed control unit 606, the profile server 604 may determine that the user has completed the “turn off lights” action.

The profile server 604 selects 624, based on the second task, a bed output event and a bed output device, wherein the bed output event comprises instructions for a bed output device, wherein the bed output device is physically coupled to a bed and capable of responding to instructions to generate some output, wherein the selected bed output event and bed output device are selected to facilitate the second task. For example, the profile server 604 may access data associated with the “turn off lights” action and identify a command to turn on another source of illumination. The profile server 604 may then access data associated with the user's profile to determine if there are any controllable sources of illumination available for the user. In this case, the user has registered an under-bed lighting system that can be controlled by the bed control unit 606. As such, the profile server 604 can generate bed output event to turn on the under-bed lighting system and transmit that command to the bed control unit 606.

The bed control unit 606 causes 626 the selected output device to perform the selected bed output event. For example, the bed control unit 606 can receive the command to turn on the under-bed lighting unit and cause the under-bed lighting unit to turn on.

The user device 602 receives 628 from the user through the computer application interface, third input indicating that the user has completed a second task. For example, after turning off the light-switch in their room and navigating back to the user device 602 using the illumination from the under-bed light, the user may provide an input to indicate that they have turned off their light-switch.

Although a particular number, order, and type of steps are described here, different numbers, orders, and types of steps may be performed to produce the same or similar results. For example, a user may wish to sleep with a humidifier running. That user, or the user's parent or guardian, may assign a humidifier output device to a ‘go to bed’ task in the user's profile. This user's routine may include the following five actions: ‘brush teeth,’ ‘wash face,’ ‘read book,’ ‘turn off lights,’ and ‘go to bed.’ The profile server 604 may track the user state as described, but may command the bed control unit 606 to engage the humidifier before the user is in the ‘go to bed’ action. For example, when the profile server 604 determines that the user is in the ‘wash face’ action, the profile server 604 may command the bed control unit 606 to engage the humidifier. As such, the humidifier will have an opportunity to begin working before it is needed for the ‘go to bed’ action.

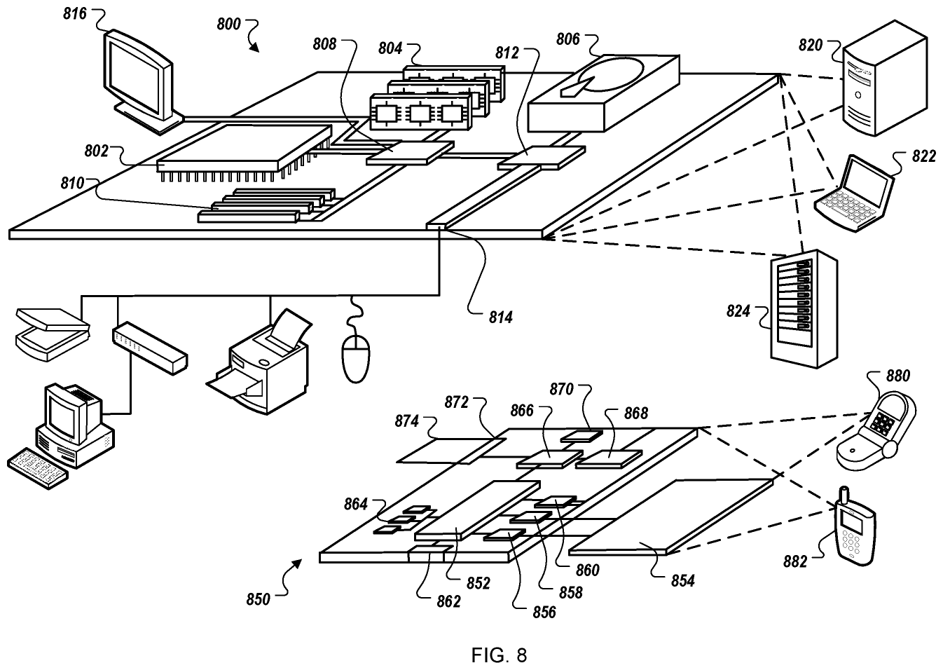

FIG. 7 is a swimlane diagram of an example process 700 for tracking a user's state and updating a profile associated with the user. In the process 700, a computer system can track the state of a user and modify data associated with that user based on a rule-set that described desired or proscribed behavior for the user. The process 700 will be described with reference to the user device 602, the profile server 604, and the bed control unit 606. However, the process 700 and other similar processes may be performed by different devices, including but not limited to devices previously described in this document.

The profile server 604 maintains 702 a user profile, the profile comprising data for a plurality of features of a computer application. For example, a user profile can include information about the user (e.g., name, age, billing information if applicable) and information about the user's accounts with various applications (e.g., which applications have been purchased or downloaded, saved game data). This profile data may be subject to privacy controls, used as part of a social network, reported to the user's parents or guardians, and handled in other ways.

The bed control unit 606 receives 704 input to a bed input device indicating at least presence of a person on the bed. For example, the bed may include a pump, pressure sensor, moisture sensor, or other sensor able to determine if a person is in the bed, as well as other information about the user. For example, the moisture sensor may be able to determine if the user has experienced a bed-wetting incident, and a light sensor may be able to determine the level of illumination around the bed. When the user enters the bed, a presence sensor may detect the user's presence and report the detection to the bed control unit 606.

The profile server 604 determines 706, from the input, a sleep parameter that represents a measure of the presence of the person on the bed. For example, the bed control unit 606 may send periodic updates about the presence of the user, alerts when the user leaves the bed, or other information. From this information, the profile server 604 can calculate one or more sleep parameters for the user. Example sleep parameters include, but are not limited to, measures reflecting duration of presence, the detection of a bed-wetting incident, a measure of restfulness of the sleep, heartbeat, and breathing.

The profile server 604 compares 708 the sleep parameter to a rule-set to determine if the sleep parameter meets a test condition of the rule-set. For example, the rule-set may have been set by the user's parent or guardian to specify that the user should remain in bed until 7:00 AM with the lights off. If the sleep parameters indicate that the user has stayed in bed until 7:00 AM with the lights off, the user has passed this test of the rule-set.

The profile server 604 modifies 710 the data for at least one of the features of the computer application. For example, if the user has met the 7:00 AM and lights off rule, content for an application in the user's profile may be released. This can include, but is not limited to, crediting the user's profile with a game resource such in-game currency, time-based access to the game, an interactive feature of the game, a badge, and an in-game statistic. If the user has not met the 7:00 AM and lights off rule, one or more alternative responses can be executed. In one example, content for an application in the user's profile may be denied, such as not debiting the user's profile with a game resource, reducing time-based access to the game, and not awarding an interactive feature of the game, a badge, and an in-game statistic. In another example, coaching-based content can be executed designed to teach and/or encourage corrected behavior, such as remaining in bed until 7:00 AM with the lights off.

The user device 602 launches 712 the application with the profile. For example, after waking up and after the user profile has been updated, the user may load their game and receive their in-game currency, play the game for the duration of their time-based access, interact with the interactive features, or view their badge or in-game statistic.

The profile server 604 generates 714 a report indicating if the sleep parameter meets the test condition of the rule-set. For example, the report may indicate the rule or rules that were examined, the data used to test the rules, and/or the results of the test. This report may take the form of a human-readable report such as text, a Portable Document Format (PDF) file, or other information intended to be read by a human reader. Alternatively or additionally, the report may take the form of a machine-readable data object. The user device 602 displays 716 the report. For example, if the report is in a human readable format, the report can be displayed to a user. If the report is in a machine-readable format, the user device 602 may render the report into a human readable format.

Although a particular number, order, and type of steps are described here, different numbers, orders, and types of steps may be performed to produce the same or similar results. For example, the user profile may be used only for a single application used to track sleep parameters.

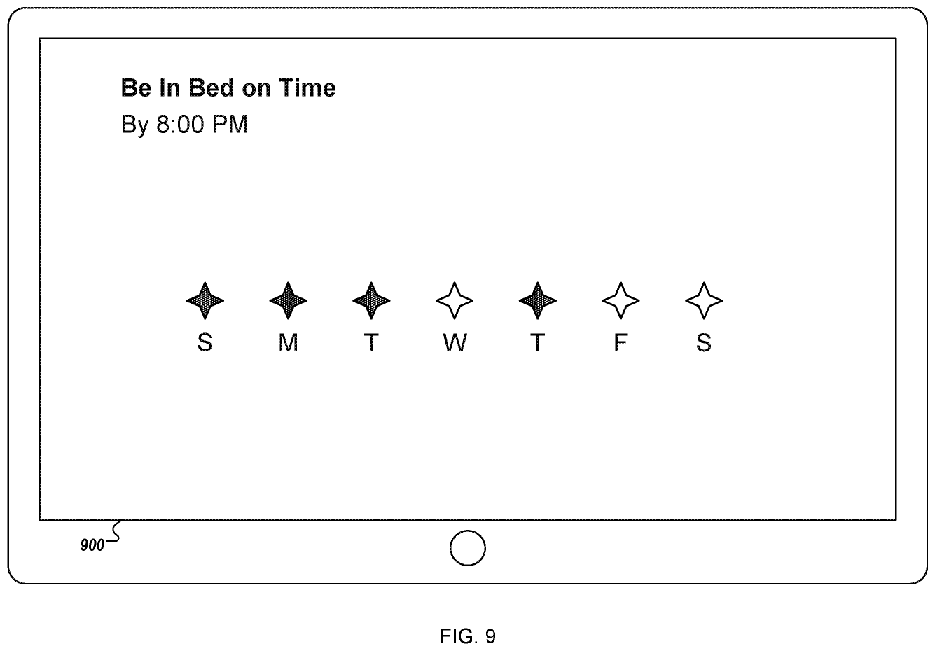

FIG. 8 shows an example of a computing device 800 and an example of a mobile computing device that can be used to implement the techniques described here. The computing device 800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, wearable computers including smart watches, and other similar computing devices. The components shown here, their connections and relationships, and their functions, are meant to be exemplary only, and are not meant to limit implementations of the inventions described and/or claimed in this document.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The computer program product can also be tangibly embodied in a computer- or machine-readable medium, such as the memory 804, the storage device 806, or memory on the processor 802.

The high-speed interface 808 manages bandwidth-intensive operations for the computing device 800, while the low-speed interface 812 manages lower bandwidth-intensive operations. Such allocation of functions is exemplary only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 864, the expansion memory 874, or memory on the processor 852. In some implementations, the computer program product can be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth, WiFi, or other such transceiver (not shown). In addition, a positioning module 870 may provide additional navigation- and location-related wireless data to the mobile computing device 850, which may be used as appropriate by applications running on the mobile computing device 850. Examples of positioning modules 870 include, but are not limited to, GPS (Global Positioning System) receiver, network triangulates, iBeacon receivers, magnetic compasses, and inertial sensors.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments of the inventions have been described. Nevertheless, it will be understood that various modifications can be made without departing from the spirit and scope of the invention. For example, in some embodiments the bed need not be adjustable. Additionally, different aspects of the different examples can be combined with other aspects as suitable for the application. Accordingly, other embodiments are within the scope of the following claims.

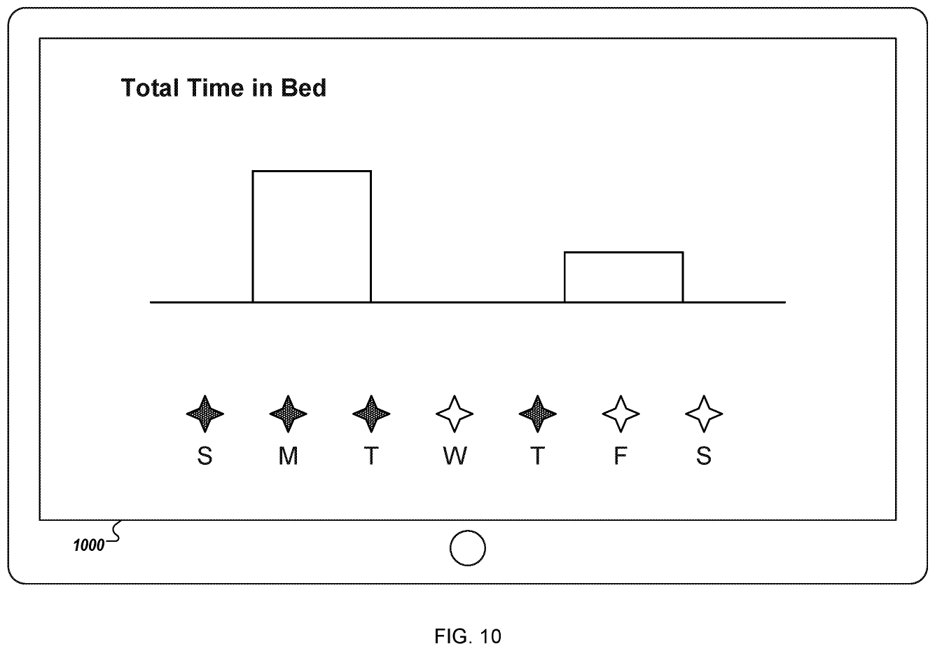

FIGS. 9 and 10 are example computer interfaces 900 and 1000 showing awards that are awarded to a user profile in response to input received from bed-based sensors. In the interfaces 900 and 1000 may be shown, for example, by a computer application that tracks the sleep habits and/or bed features associated with one or more users. For example, interfaces 900 and 1000 may be used by one of the applications previously described, as part of a webpage or application provided by the manufacturer of a bed previously described, and/or may be used as part of a system for launching one of the applications previously described.

In the interface 900, a report is displayed that shows the results of tracking a user's time in bed. In this example, the user's bedtime is set to 8:00 PM. This user bedtime may be set by the user, or by another person that is, for example, responsible for or assisting the user. Such other person may be, for example, the user's parent or guardian.

When the user enters the bed, the user's presence may be identified, as has been previously described. For example, a pressure or other sensor in the bed may receive input indicating that the user has entered the bed. The timing of this entry event may be identified, for example by a controller of the bed or a computer, and compared with the user's bedtime.

If the entry event occurs before the user's bedtime, the system may determine that the user has gone to bed by their bedtime, and a profile associated with the user, and comprising data for a plurality of features of a computer application, may be modified to reflect the user's success in going to bed by their bed time.

As previously described, the user may be given access to features of a video game or other application. In addition or in the alternative, the user may be given awards in the profile shown in the interface 900. In this example, the user is given a “star” for each night that they go to bed by the bedtime listed in their account. As shown, the user has earned as tar for Sunday, Monday, Tuesday, and Thursday, but not for Wednesday. The represents the users going to bed by their bed time on Sunday, Monday, Tuesday, and Thursday, but not Wednesday.

While stars are shown in the example shown here, other types of awards may be used. For example, a badge may be given in response to meeting a single night's bedtime, or meeting some other rule (e.g., earning seven consecutive stars). In another example, a graphical element may be shown to grow or mature (e.g., a character may produce a thought bubble showing their dreams that develop the longer the character sleeps).

In the example interface 1000, a similar set of star awards are shown in an interface that includes other user-related data. In this example, the sleep awards stars are shown a bar graph showing the user's total time in bed. In other example, other information may be included, for example to provide a viewer with other information with which to understand the sleep habits of the user.

1.-12. (canceled)

13. A system comprising:
an articulation controller configured to adjust a position of a bed;
one or more processors; and
a memory storing instructions, wherein the instructions, when executed by the one or more processors, cause the one or more processors to perform operations comprising:
receiving a user input including at least one wakeup time and at least one sleep time;
adjusting the position of the bed to a wakeup position at the at least one wakeup time using the articulation controller; and
adjusting the position of the bed to a sleep position at the at least one sleep time using the articulation controller.

14. The system of claim 13, wherein each of the at least one wakeup time and the at least one sleep time is set for one or more days of each week.

15. The system of claim 13, wherein adjusting the position of the bed to the wakeup position or the sleep position comprises adjusting the position of the bed by adjusting an adjustable foundation that supports the bed.

16. The system of claim 13, wherein adjusting the position of the bed to the wakeup position or the sleep position comprises adjusting a position of one or more portions of the bed to the wakeup position or the sleep position.

17. The system of claim 16, wherein adjusting the position of the one or more portions of the bed to the wakeup position or the sleep position comprises adjusting a position of a first portion of the bed to the wakeup position or the sleep position, while maintaining a position of a second portion of the bed.

18. The system of claim 16, wherein adjusting the position of the one or more portions of the bed to the wakeup position or the sleep position comprises adjusting a position of a first portion of the bed for a first user to the wakeup position or the sleep position, while maintaining a position of a second portion of the bed for a second user.

19. The system of claim 13, the operations further comprise vibrating the bed at the at least one wakeup time or the sleep time.

20. The system of claim 13, the operations further comprise implementing a sunrise lighting scheme at the at least one wakeup time and implementing a sunset lighting scheme at the at least one sleep time in a bedroom where the bed is located.

21. A system comprising:
one or more processors; and
a memory storing instructions, wherein the instructions, when executed by the one or more processors, cause the one or more processors to perform operations comprising:
receiving a user input including at least one wakeup time and at least one sleep time;
adjusting a position of a bed to a wakeup position at the at least one wakeup time; and
adjusting the position of the bed to a sleep position at the at least one sleep time.

22. The system of claim 21, wherein each of the at least one wakeup time and the at least one sleep time is set for one or more days of each week.

23. The system of claim 21, wherein adjusting the position of the bed to the wakeup position or the sleep position comprises adjusting the position of the bed by adjusting an adjustable foundation that supports the bed.

24. The system of claim 21, wherein adjusting the position of the bed to the wakeup position or the sleep position comprises adjusting a position of one or more portions of the bed to the wakeup position or the sleep position.

25. The system of claim 24, wherein adjusting the position of the one or more portions of the bed to the wakeup position or the sleep position comprises adjusting a position of a first portion of the bed to the wakeup position or the sleep position, while maintaining a position of a second portion of the bed.

26. The system of claim 24, wherein adjusting the position of the one or more portions of the bed to the wakeup position or the sleep position comprises adjusting a position of a first portion of the bed for a first user to the wakeup position or the sleep position, while maintaining a position of a second portion of the bed for a second user.

27. The system of claim 21, the operations further comprise vibrating the bed at the at least one wakeup time or the sleep time.

28. The system of claim 21, the operations further comprise implementing a sunrise lighting scheme at the at least one wakeup time and implementing a sunset lighting scheme at the at least one sleep time in a bedroom where the bed is located.

29. A method comprising:
receiving a user input including at least one wakeup time and at least one sleep time;
adjusting a position of a bed to a wakeup position at the at least one wakeup time using an articulation controller; and
adjusting the position of the bed to a sleep position at the at least one sleep time using the articulation controller.

30. The method of claim 29, wherein adjusting the position of the bed to the wakeup position or the sleep position comprises adjusting a position of one or more portions of the bed to the wakeup position or the sleep position.

31. The method of claim 30, wherein adjusting the position of the one or more portions of the bed to the wakeup position or the sleep position comprises adjusting a position of a first portion of the bed to the wakeup position or the sleep position, while maintaining a position of a second portion of the bed.

32. The method of claim 29, the method further comprises implementing a sunrise lighting scheme at the at least one wakeup time and implementing a sunset lighting scheme at the at least one sleep time in a bedroom where the bed is located.
